(19) United States
(12) Patent Application Publication
(10) Pub. No.: US 2024/0309403 A1
(43) Pub. Date: Sep. 19, 2024

(54) CELL PROCESSING METHOD AND CELL PROCESSING APPARATUS

(71) Applicant: CANON KABUSHIKI KAISHA, Tokyo (JP)

(72) Inventors: TATSUAKI ORIHARA, Tokyo (JP); YOSHINORI ITOH, Tokyo (JP); FUTOSHI HIROSE, Kanagawa (JP); SACHIKO YAMAUCHI, Kanagawa (JP); YUKI MORITA, Tokyo (JP)

(21) Appl. No.: 18/676,661

(22) Filed: May 29, 2024

Related U.S. Application Data
(63) Continuation of application No. PCT/JP2022/045106, filed on Dec. 7, 2022.

(30) Foreign Application Priority Data
Dec. 9, 2021 (JP) 2021-200174
Dec. 1, 2022 (JP) 2022-193111

(51) Int. Cl. C12N 15/89

(52) U.S. Cl. CPC C12N 15/895

(57) ABSTRACT

The cell processing method includes a step of passing a liquid containing a cell through an orifice from a flow path by a pressure applying unit, and is characterized in that the orifice is connected to the flow path so as to narrow a flow from the flow path and in that the orifice includes at least any one of a portion in which an area of a cross-section perpendicular to a flow direction of the liquid passing through a center of the orifice is prevented from being changed from an inlet toward an outlet of the orifice or a portion in which the area is increased from the inlet toward the outlet of the orifice. The cell processing apparatus is characterized by including an orifice forming member having the orifice, the flow path, and the pressure applying unit.

### CROSS-REFERENCE TO RELATED APPLICATIONS

This application is a Continuation of International Patent Application No. PCT/JP2022/045106, filed Dec. 7, 2022, which claims the benefit of Japanese Patent Application No. 2021-200174, filed Dec. 9, 2021, and Japanese Patent Application No. 2022-193111, filed Dec. 1, 2022, all of which are hereby incorporated by reference herein in their entirety.

### BACKGROUND OF THE INVENTION

### Field of the Invention

The present invention relates to a cell processing method and a cell processing apparatus.

### Description of the Related Art

Along with the advancement of biotechnology, cell processing as typified by gene introduction is becoming increasingly important. As a method for gene introduction that is a kind of cell processing, a chemical method using a cationic chemical substance or the like, a biological method using a virus, and a physical method such as microinjection or electroporation are generally known. Any of these methods have advantages and disadvantages and are often selected and used in accordance with the application.

In Japanese Patent No. 5645657, as the physical method, there is a description of a method of introducing a gene by passing a cell suspension through an orifice.

In the method described in Japanese Patent No. 5645657, the efficiency of gene introduction is not sufficient, and there is a demand for further improvement. In addition, even when the method of Japanese Patent No. 5645657 is applied to cell processing, such as perforation of a cell membrane, introduction of a target substance not limited to a nucleic acid, and cell lysis, the efficiency is not sufficient, and there is a demand for further improvement.

The present invention has been made in view of the above-mentioned problems, and an object of the present invention is to provide a cell processing method and a cell processing apparatus having high efficiency of cell processing.

### SUMMARY OF THE INVENTION

The above-mentioned object is achieved by the cell processing method and the cell processing apparatus according to the present invention.

That is, according to the present invention, there is provided a cell processing method including a step of passing a liquid containing a cell through an orifice from a flow path by a pressure applying unit, wherein the orifice is connected to the flow path so as to narrow a flow from the flow path, and wherein the orifice includes at least any one of a portion in which an area of a cross-section perpendicular to a flow direction of the liquid passing through a center of the orifice is prevented from being changed from an inlet toward an outlet of the orifice or a portion in which the area is increased from the inlet toward the outlet of the orifice.

In addition, according to the present invention, there is provided a cell processing apparatus including: an orifice forming member having an orifice; a flow path connected to the orifice; and a pressure applying unit, wherein the pressure applying unit is configured to enable a flow of a liquid containing a cell directed from the flow path toward the orifice to be generated, wherein the orifice is configured to narrow the flow of the liquid from the flow path, and wherein the orifice includes at least any one of a portion in which an area of a cross-section perpendicular to a flow direction of the liquid passing through a center of the orifice is prevented from being changed from an inlet toward an outlet of the orifice or a portion in which the area is increased from the inlet toward the outlet of the orifice.

Further features of the present invention will become apparent from the following description of exemplary embodiments with reference to the attached drawings.

### BRIEF DESCRIPTION OF THE DRAWINGS

FIG. 1A is a schematic view for illustrating the configuration of a cell processing apparatus according to a first embodiment of the present invention.

FIG. 1B is a schematic view for illustrating an orifice plate to be used in the cell processing apparatus according to the first embodiment of the present invention.

FIG. 1C is a sectional view for illustrating the shape of an orifice included in the orifice plate illustrated in FIG. 1B.

FIG. 2A is a schematic view for illustrating one form of a thermal inkjet type liquid ejection head that may be suitably used in the present invention.

FIG. 2B is an enlarged view for illustrating a cross-section taken along the line E-E of a pressure element substrate included in the thermal inkjet type liquid ejection head illustrated in FIG. 2A.

### DESCRIPTION OF THE EMBODIMENTS

Cell processing in the present invention refers to causing some transient or persistent change in a cell. Examples of the cell processing include perforation (sometimes referred to as “poration”) of a cell membrane involving opening a pore in a cell membrane, introduction of a target substance into a cell, and lysis that destroys a cell, but the cell processing is not limited thereto as long as the cell processing is in line with the main purpose of the present invention.

The mechanism by which a target substance such as a gene is introduced into a cell has not been fully elucidated, but it is assumed that transient perforation occurs in a cell membrane to cause the introduction of a target substance that is present in the surrounding area. The target substance is not driven by electrophoresis, and hence, for example, a nonionic substance (neutral substance) such as dextran can also be introduced without depending on the charge amount of the target substance. In addition, a substance that may be denatured by an electric field, such as a protein, can also be introduced without such concern. The size of a pore that can be opened is estimated to be 100 nm or more based on the size of a substance to be introduced, and thus a considerably large substance can also be introduced. The mechanism by which a cell is lysed has not been fully elucidated, either, but it is assumed that a cell is irreversibly destroyed when perforation of a cell membrane occurs so strongly that survival cannot be maintained.

It is known that, in general, when a liquid passes through an orifice, a strong shear stress is generated because of, for example, a change in flow velocity at the time of passage of the liquid or a difference in flow velocity depending on the position at which the liquid passes through an inlet of the orifice. It is considered that, when the liquid contains a cell, the cell is subjected to a stress from the liquid, which causes various kinds of cell processing such as the perforation of a cell membrane. According to the analysis of the inventors, the nozzles of HP51629a and HP51626a cartridges described in Japanese Patent No. 5645657 each had a shape that was narrowed while having a curved surface from an inlet to an outlet, and the area of the inlet was larger than that of the outlet. That is, the cross-section perpendicular to a flow direction of a liquid passing through the center of an orifice was reduced in size from the inlet toward the outlet of the orifice.

The flow velocity is gently increased in a portion in which the cross-section perpendicular to the flow direction of the liquid in the orifice is reduced in size. Meanwhile, in the present invention, the orifice includes at least any one of the following two portions. One of the above-mentioned two portions is a portion in which the area of the cross-section perpendicular to the flow direction of the liquid passing through the center of the orifice is prevented from being changed from the inlet toward the outlet of the orifice. In addition, the other of the above-mentioned two portions is a portion in which the area is increased from the inlet toward the outlet of the orifice. Thus, as compared to the case including only the portion in which the cross-section is reduced in size, the orifice includes a portion in which a change speed of the flow velocity is large. It is conceived that the foregoing serves as a factor for further enhancing the efficiency of cell processing. In addition, the orifice may include both the portion in which the area of the cross-section perpendicular to the flow direction of the liquid passing through the center of the orifice is prevented from being changed from the inlet toward the outlet of the orifice and the portion in which the area is increased from the inlet toward the outlet of the orifice.

The cell processing method and the cell processing apparatus according to the present invention are described in detail below by illustrating embodiments. However, the configurations, structures, materials, settings and the like to be used in the following embodiments are to be appropriately changed in accordance with various conditions under which the present invention is applied, and are not intended to limit the scope of the present invention.

### First Embodiment

The cell processing apparatus according to the present invention includes an orifice forming member having an orifice; a flow path connected to the orifice; and a pressure applying unit. The pressure applying unit is configured to enable a flow of a liquid containing a cell directed from the flow path toward the orifice to be generated. In addition, the orifice is configured to narrow the flow of the liquid from the flow path, and further, the orifice includes at least any one of the following two portions. One of the above-mentioned two portions is a portion in which the area of the cross-section perpendicular to the flow direction of the liquid passing through the center of the orifice is prevented from being changed from the inlet toward the outlet of the orifice. In addition, the other of the above-mentioned two portions is a portion in which the area is increased from the inlet toward the outlet of the orifice.

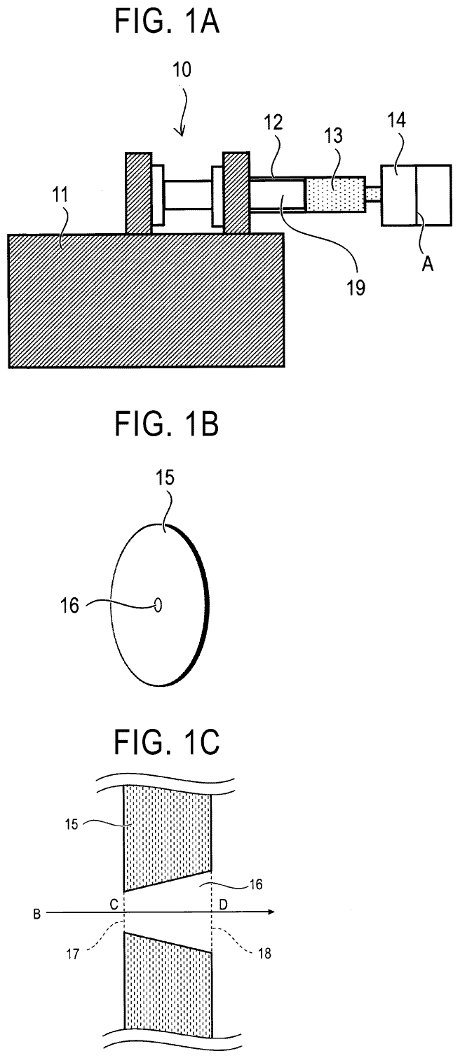

FIG. 1A is a schematic view for illustrating the configuration of a cell processing apparatus according to a first embodiment of the present invention. A cell processing apparatus 10 according to the first embodiment of the present invention includes a holder 14, a syringe 12, a piston 19, and a syringe pump 11.

The syringe 12 has a cylindrical shape and can accommodate therein a liquid 13 containing a cell to be processed. The syringe 12 functions as a part of a flow path leading to the inside of the holder 14 coupled to the syringe 12 at the time of cell processing. The holder 14 is formed of two parts, and an orifice plate 15 serving as an orifice forming member illustrated in FIG. 1B is fixed and encapsulated in a region A between the two parts.

The orifice plate 15 has an orifice 16 as illustrated in FIG. 1B. The orifice refers to a small hole through which a fluid flows, and an orifice plate refers to a thin plate having an orifice. A flow path leading from the syringe 12 to the inside of the holder 14 formed at the time of cell processing is connected to the orifice 16. In this case, the flow path immediately before the orifice 16 is larger than an opening of an inlet of the orifice 16. That is, the orifice 16 is configured to narrow the flow of the liquid 13 containing a cell from the flow path leading from the syringe 12 to the inside of the holder 14.

FIG. 1C is a sectional view for illustrating the shape of the orifice 16 included in the orifice plate 15, illustrating a cross-section along a plane including the center of the orifice 16 and being parallel to the flow direction of the liquid containing a cell that passes through the center of the orifice. The flow of the liquid 13 containing a cell from the flow path leading from the syringe 12 to the inside of the holder 14 enters the orifice 16 through an inlet 17 and exits therefrom through an outlet 18. As illustrated in FIG. 1C, in the orifice 16, the cross-section perpendicular to a flow direction B of the liquid passing through the center of the orifice 16 monotonically expands from the inlet 17 toward the outlet 18 of the orifice 16.

When the cell processing is performed through use of the cell processing apparatus 10, the syringe pump 11 serving as a pressure generating unit is operated to push the piston 19 serving as the pressure applying unit into the syringe 12. Thus, a flow of the liquid 13 containing a cell directed from the flow path connected to the orifice 16 toward the orifice 16 can be generated. The liquid 13 containing a cell enters the holder 14 from the syringe 12 and passes through the orifice 16 included in the orifice plate 15 fixed inside the holder 14. The cell in the liquid 13 containing a cell is processed in the process of entering the orifice 16 through the inlet 17 to exiting therefrom through the outlet 18.

When the syringe pump 11 is driven, the liquid 13 containing a cell that has passed through the orifice 16 flows out from an outlet of the holder 14 and is received by a dish or the like. When the force of the liquid containing a cell is strong, the liquid linearly flies out. Thus, it is preferred that a way of arranging the cell processing apparatus 10 and the dish be devised so that the liquid does not protrude or fly out.

### <Liquid Containing Cell>

The liquid 13 containing a cell is a liquid containing at least a cell to be processed. The cell is preferably suspended in a liquid. That is, the liquid containing a cell is preferably a cell suspension.

### (Kind of Cell)

The cell is not particularly limited and may be an adherent cell, a floating cell, a spheroid that is an aggregate thereof, or the like. The cell may be a cell line or a primary cell.

The cell may be any one of a eukaryotic cell or a prokaryotic cell, and examples thereof include mammalian cells, insect cells, plant cells, yeast cells, and Escherichia coli.

### (Diameter of Cell)

The diameter of a cell may be measured by placing the liquid 13 containing a cell on a hemocytometer or the like, and performing measurement using, for example, an optical microscope mounted with an image sensor. Through use of an image recorded using the image sensor, the diameter of a cell may be determined in accordance with purposes based on distance information corresponding to an image stored in advance. It is preferred that the cell be brought into focus, and then an imaging image be recorded, followed by length measurement. When there is a variation in diameter of the cell, a number average value is defined as the diameter of the cell.

### (Liquid)

A liquid is not particularly limited, but examples thereof include water and physiological saline. Further, examples include buffers, such as phosphate buffered saline (hereinafter referred to as “PBS”) and Tris. In addition, examples include various media, such as Dulbecco's Modified Eagle Medium (hereinafter referred to as “D-MEM”), Iscove's Modified Dulbecco's Medium (hereinafter referred to as “IMDM”), Hanks' Balanced Salt Solutions (hereinafter referred to as “HBSS”), Minimum Essential Medium-Eagle, Earle's Salts Base, with Non-Essential Amino Acid (hereinafter referred to as “MEM-NEAA”), Roswell Park Memorial Institute Medium (RPMI) 1640, and F-12. In addition, sera, commercially available buffers for electroporation, commercially available buffers for FACS analysis, or infusion solutions such as lactated Ringer's solution can be used as the liquid. Two or more kinds of those liquids may be used as a mixture. The water is preferably water that has been deionized by ion exchange or the like, and sterilized with an autoclave or the like.

The liquid 13 containing a cell may contain a target substance to be introduced into the cell by processing. The target substance to be introduced can be appropriately selected in accordance with purposes. Examples thereof include a nucleic acid, a protein, and a labeling substance, but the target substance is not limited thereto. When the liquid 13 containing a cell is not allowed to contain the target substance to be introduced into the cell, it is only required that the cell be brought into contact with a liquid containing a target substance after passing through the orifice. For example, there is a method involving placing a liquid containing a target substance in advance in a dish serving as a receiving tray when a cell suspension is taken out from the orifice.

### (Nucleic Acid)

For the purpose of transiently and stably expressing a nucleic acid or interfering with a gene, an exogenous ribonucleic acid (RNA) or deoxyribonucleic acid (DNA) that is different from those derived from the cell into which the nucleic acid is introduced may be used as the introduction compound. As a higher-order structure that the nucleic acid may have, there are given a single-stranded primary structural body, secondary structures, such as a hairpin-shaped stem-loop structure and a helix structure. In addition, the nucleic acid may also have a higher-order structure of a tertiary structure such as A-Form, B-Form, or Z-Form. Further, as the nucleic acid, a nucleic acid having a quaternary structure such as a supercoiled shape may be used. The nucleic acids having these higher-order structures may be suitably used in accordance with purposes. In addition, as the nucleic acid, a nucleic acid labeled with a fluorescent compound or a radioactive isotope may be used in accordance with purposes.

Examples of the RNA include messenger RNA, which serves to copy and transport a sequence from DNA to a ribosome, which is an organelle for synthesizing a protein. In addition, ribosomal RNA, which is a constituent element of the ribosome, or transfer RNA, which transfers an amino acid having a sequence corresponding to a sequence of the ribosomal RNA to the ribosome, may be used as the RNA to be introduced into a cell. Other examples of the RNA include small nuclear RNA, small nucleolar RNA, microRNA, and siRNA having an interference action. However, the RNA is not limited to the foregoing, and suitable RNA may be used in accordance with purposes.

As the DNA, any of single-stranded DNA, double-stranded DNA, triple-stranded DNA, and quadruplex DNA may be selected and used. With regard to the shape of the DNA, a linear shape, a circular shape, or the like is generally used, but the DNA may have any shape such as DNA origami, which has been attracting attention in recent years, and the shape of the DNA is not particularly limited. As the DNA to be introduced, double-stranded DNA is preferred in terms of stability, and circular plasmid DNA is more preferred in terms of the ease of amplification in Escherichia coli or yeast. Further, to be introduced into a cell, the DNA needs to be introduced from above the cell membrane into the inside of the cell, and hence preferably has as small a surface area as possible. For example, even among DNAs having identical sequences, compared to linear DNA, circular DNA is preferred, and supercoiled DNA resulting from the twisting of DNA is more preferred.

### (Protein)

Examples of the protein include a protein dissolved or dispersed in a liquid containing a cell, or dispersed in a state of being supported on a substrate. Examples of the structure that the protein has include: a primary structure including a polypeptide; secondary structures, such as an α-helix and a β-sheet; a tertiary structure containing those secondary structures; and a quaternary structure such as hemoglobin. The structure that the protein has is not particularly limited, and it is only required that the protein have a structure in accordance with purposes. Specific examples of the protein include: enzyme proteins such as amylase; structural proteins, such as collagen and keratin; transport proteins such as albumin; storage proteins such as ferritin; contractile proteins, such as actin and myosin; protective proteins such as globulin; regulatory proteins such as calmodulin; other various membrane proteins; zinc finger nucleases for genome editing; and a Cas9 protein used for CRISPR/Cas9.

### (Labeling Substance)

Examples of the labeling substance include the one obtained by chemically or physically modifying the nucleic acid, the protein, or the like mentioned above to allow labeling to be recognized from the outside of a cell after being introduced into the cell. The labeling substance may have such an absorption wavelength or fluorescence wavelength as to be recognizable separately from the cell into which the labeling substance is introduced. In addition, the labeling substance may be allowed to be present in a state of being dissolved or dispersed in a solution, or dispersed while being supported on a substrate. Specific examples of the labeling substance include a stable isotope substance, such as deuterium, 13C, or 15N, a radioactive substance, a dye, a fluorescent dye, a pigment, a fluorescent pigment, quantum dots, nanodiamond, fullerene, a carbon nanosheet, and a carbon nanotube.

The liquid 13 containing a cell may appropriately contain other components in addition to the cell and the target substance. Examples of the other components include a salt, a saccharide, a ribonucleotide, a growth factor, a hormone, a pH buffering agent, a surfactant, a chelating agent, a water-soluble organic solvent, a protein, an amino acid, an antimicrobial agent, a moisturizing agent, and a thickener.

### (Salt)

Examples of the salt include inorganic salts and organic salts to be used for cell culture. Specifically, examples of the salt include sodium chloride, potassium chloride, and sodium citrate.

### (Saccharide)

Examples of the saccharide include glucose, sucrose, and fructose. Those saccharides may be used for the purpose of supplying a nutrient to cells, adjusting an osmotic pressure, or the like.

### (Ribonucleotide)

Examples of the ribonucleotide include adenosine triphosphate and guanosine triphosphate. Those ribonucleotides may be used for the purpose of aiding cell metabolism.

### (Growth Factor and Hormone)

Examples of the growth factor and the hormone include a human growth hormone, other animal growth hormones, such as a bovine growth factor, a porcine growth factor, and a chicken growth factor, insulin, oxytocin, angiotensin, methionine enkephalin, substance P, ET-1, FGF, KGF, EGF, IGF, PDGF, LHRH, GHRH, FSH, DDAVP, PTH, vasopressin, glucagon, and somatostatin.

### (pH Buffering Agent)

Examples of the pH buffering agent include a citrate buffer, a phosphate buffer, a Tris buffer, and a HEPES buffer.

### (Surfactant)

Examples of the surfactant include anionic, cationic, amphoteric, and nonionic water-soluble surfactants. One kind or a plurality of kinds of those surfactants may be used.

### (Chelating Agent)

Examples of the chelating agent include ethylenediaminetetraacetic acid (EDTA) and ethylene glycol tetraacetic acid (EGTA).

### (Water-Soluble Organic Solvent)

Examples of the water-soluble organic solvent may include glycerin, polyethylene glycol, and dimethyl sulfoxide. The content of the water-soluble organic solvent in the liquid 13 containing a cell is preferably 0.001 mass % or more and 50 mass % or less with respect to the total mass of the liquid 13 containing a cell.

### (Protein and Amino Acid)

Examples of the protein and the amino acid include sera, such as fetal bovine serum (hereinafter referred to as “FBS”) and horse serum.

### (Antimicrobial Agent)

Examples of the antimicrobial agent include an antibiotic such as penicillin streptomycin and sodium azide.

### (Moisturizing Agent)

Examples of the moisturizing agent include polyhydric alcohols, such as glycerin, propylene glycol, butylene glycol, and sorbit. Further, examples include mucopolysaccharides, such as hyaluronic acid and chondroitin sulfate, soluble collagen, and hydrolysates of proteins, such as elastin and keratin. Those moisturizing agents may be used alone or as a mixture thereof.

### (Thickener)

Examples of the thickener include: starches, such as an oxidation-modified starch, an enzyme-modified starch, a thermochemically modified starch, a cationic starch, an amphoteric starch, and an esterified starch. In addition, further examples of the thickener include: cellulose derivatives, such as carboxymethyl cellulose, hydroxyethyl cellulose, and ethyl cellulose; and natural or semi-synthetic polymers, such as casein, gelatin, and a soy protein. Another example of the thickener is a fully or partially saponified water-soluble polymer compound. Examples of the water-soluble polymer compound include polyvinyl alcohols, such as polyvinyl alcohol, acetoacetylated polyvinyl alcohol, carboxy-modified polyvinyl alcohol, olefin-modified polyvinyl alcohol, and silyl-modified polyvinyl alcohol. Of those, at least one water-soluble polymer compound may be appropriately selected and used.

### <Orifice>

The material for the orifice plate 15 serving as the orifice forming member is not particularly limited, but a metal, a resin, or the like may be suitably used. The orifice 16 may be formed by suitably using laser processing or etching although there is no particular limitation. An example in which the orifice plate 15 has one orifice 16 is illustrated in FIG. 1B, but a plurality of orifices 16 may be formed.

The sectional shape of the orifice perpendicular to the flow direction B of the liquid passing through the center of the orifice is preferably substantially circular as illustrated in FIG. 1B, but the sectional shape may be any shape such as an oval shape, a polygonal shape, or a shape having a protrusion.

In this embodiment, the opening of the inlet 17 of the orifice 16 is formed to be smaller than the opening of the outlet 18. Here, the inlet 17 refers to an opening portion on an upstream side of the flow of the liquid 13 containing a cell, and the outlet 18 refers to an opening portion on a downstream side of the flow. In the present invention, the orifice includes at least any one of the following two portions. One of the above-mentioned two portions is a portion in which the area of the cross-section perpendicular to the flow direction of the liquid passing through the center of the orifice is prevented from being changed from the inlet toward the outlet of the orifice. In addition, the other of the above-mentioned two portions is a portion in which the area is increased from the inlet toward the outlet of the orifice. Thus, for example, the opening areas of the inlet and the outlet of the orifice may be the same, and the shape of the cross-section perpendicular to the flow direction of the liquid passing through the center of the orifice may be constant inside the orifice. Alternatively, the opening of the inlet of the orifice may be smaller than the opening of the outlet 18, and the contour line connecting the inlet and the outlet to each other in the cross-section of the orifice parallel to the direction from the inlet to the outlet of the orifice may be a curve.

The opening area of the inlet in the portion in which the above-mentioned area is increased from the inlet toward the outlet of the orifice may be less than 1.0 times the opening area of the outlet in this portion, but is more preferably less than 0.8 times.

The cell is basically processed by the action from the liquid, and hence the cell is not necessarily required to be brought into contact with a wall surface of the orifice. Cell processing may be performed by bringing the cell into contact with the wall surface, but there easily arises a problem in that the orifice is inevitably narrowed to cause cell clogging. Thus, it is preferred that the width of the narrowest portion of the cross-section perpendicular to the flow direction of the liquid passing through the center of the orifice be larger than the diameter of the cell. In addition, the inlet circle-equivalent diameter of each of the above-mentioned two portions is preferably 10 times or less the diameter of the cell. When the pore diameters of the two portions are large, the force which the cell receives when passing through the two portions becomes weak, and the efficiency of cell processing is decreased. The inlet circle-equivalent diameters of the two portions are each preferably 1 μm or more and 100 μm or less. The inlet circle-equivalent diameter as used herein refers to the diameter of a circle having an area equal to the opening area of the inlet. The distance from the inlet to the outlet of each of the two portions is not limited, but is preferably 1 μm or more and 200 μm or less. When the distance from the inlet to the outlet of each of the two portions is long, the pressure loss is increased, and a large pressure is required for passing the cell suspension.

### (Holder)

As described in this embodiment, it is convenient that the orifice plate 15 be mounted on the holder 14 that supports the orifice plate 15 and is sealed so that the liquid does not flow out to a place other than the orifice. When the orifice plate 15 is fixed and encapsulated by the holder 14, deformation of the orifice plate can be prevented even when the orifice plate is thin. In addition, when the orifice plate 15 is fixed and encapsulated by the holder 14, the flow path leading from the syringe 12 to the inside of the holder 14 can be reliably connected to the orifice.

### (Syringe)

The kind of the syringe 12 is not limited, and examples thereof include plastic syringes and glass syringes. Of those, a gas-tight syringe is particularly preferred. The orifice 16 is small and has a large pressure loss, and hence deformation of the syringe 12 and leakage of the liquid 13 containing a cell from the syringe 12 may occur. The gas-tight syringe has high rigidity and high airtightness, and hence the liquid 13 containing a cell can be stably pushed out.

When a combination of the syringe 12 and the holder 14 with a locking function is used, leakage of the liquid 13 containing a cell from the coupled site between the syringe 12 and the holder 14 can be suppressed. It is preferred that, before the syringe 12 and the holder 14 are coupled to each other, the space from the coupled site between the syringe 12 and the holder 14 to the orifice plate be filled with the liquid 13 containing a cell in advance. With this configuration, air can be prevented from obstructing the entrance of the liquid 13 containing a cell into the orifice 16, and the flow velocity of the liquid 13 containing a cell can be stabilized.

### (Syringe Pump)

The syringe pump 11 is not particularly limited as long as the syringe pump 11 can generate a pressure for pushing the piston 19 into the syringe 12. However, in order to obtain a desired flow rate, a syringe pump having a sufficient driving force and a sufficient driving speed is preferably used.

### Other Embodiments

The present invention can be carried out without limitation within a scope not departing from the gist. That is, the present invention can be carried out without limitation as long as the present invention includes a step of passing the liquid containing a cell through the orifice specified by the present invention from the flow path by the pressure applying unit.

In the first embodiment, there is described an example in which the liquid containing a cell is passed through the orifice through use of the syringe forming a part of the flow path, the syringe pump serving as the pressure generating unit, and the piston serving as the pressure applying unit. However, as a member forming a part of the flow path, for example, a glass tube, a tube, or the like may be used instead of the syringe, and any member may also be used as the member forming a part of the flow path as long as a flow introducing the liquid containing a cell into the orifice can be formed. In addition, also as a member to be used as the pressure generating unit, various pumps, actuators, and the like may be used instead of the syringe pump. As the pressure applying unit, any member may be used as long as the member can apply a pressure for passing the liquid containing a cell through the orifice. In addition, the pressure applying unit may also function as the pressure generating unit. For example, a heating element included in a thermal inkjet type liquid ejection head and a piezoelectric element included in a piezoelectric inkjet type liquid ejection head described later, which are pressure generating units, also function as pressure generating units.

Further, as the cell processing apparatus according to the present invention, an inkjet type liquid ejection head may be suitably used. The kind of the inkjet type liquid ejection head is not limited. A piezoelectric inkjet type liquid ejection head or a thermal inkjet type liquid ejection head may be used.

The thermal inkjet type liquid ejection head that may be suitably used as the cell processing apparatus in the present invention is described.

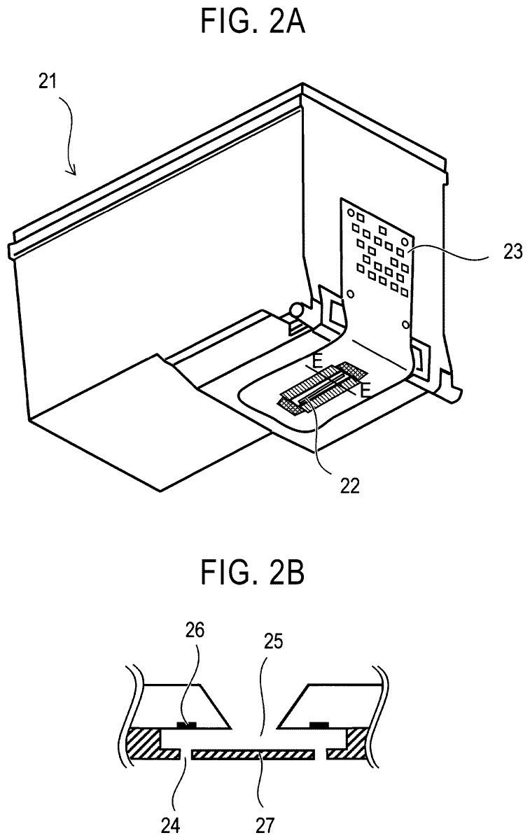

FIG. 2A is a schematic view for illustrating one form of the thermal inkjet type liquid ejection head. A thermal inkjet type liquid ejection head 21 includes a pressure element substrate 22, an electrical connection portion 23 that sends electric power or a signal to the pressure element substrate 22 from an inkjet printer or the like, and a space capable of holding a liquid to be ejected. FIG. 2B is an enlarged view for illustrating a cross-section taken along the line E-E of the pressure element substrate 22 included in the thermal inkjet type liquid ejection head 21 illustrated in FIG. 2A.

The pressure element substrate 22 includes orifices 24 for ejecting a liquid containing a cell, a flow path 25 that supplies the liquid containing a cell to the orifices 24, heating elements (electrothermal conversion elements) 26 each serving as a pressure applying unit that applies a pressure for ejecting the liquid containing a cell, and a flow path forming member 27. The flow path forming member 27 forms the flow path 25. The flow path forming member 27 also serves as the orifice forming member according to the present invention and has the orifices 24. When the heating element 26 is energized for a short period of time, the liquid in the vicinity of the heating element 26 is foamed, and the liquid containing a cell passes through the orifice 24 to be ejected.

The orifice 24 included in the pressure element substrate 22 includes at least any one of the following two portions. One of the above-mentioned two portions is a portion in which the area of the cross-section perpendicular to the flow direction of the liquid passing through the center of the orifice 24 is prevented from being changed from the inlet toward the outlet of the orifice 24. In addition, the other of the above-mentioned two portions is a portion in which the area is increased from the inlet toward the outlet of the orifice 24. The cell is processed with high efficiency in a process in which the liquid containing a cell passes through the orifice 24 from the flow path 25 to be ejected. In addition, the orifice 24 may include both the portion in which the area of the cross-section perpendicular to the flow direction of the liquid passing through the center of the orifice is prevented from being changed from the inlet toward the outlet of the orifice and the portion in which the area is increased from the inlet toward the outlet of the orifice.

The thermal inkjet type liquid ejection head is described as an example, but the pressure applying unit is a piezoelectric element in the case of the piezoelectric inkjet type liquid ejection head. When the piezoelectric element is energized for a short period of time, the piezoelectric element is deformed, and the liquid containing a cell passes through the orifice to be ejected.

### EXAMPLES

The present invention is specifically described below by way of Examples and Comparative Examples. The present invention is not limited in any way by the following Examples within a scope not departing from the gist.

### Examples 1 to 6

In each of Examples 1 to 6, a target substance was introduced through use of the cell processing apparatus illustrated in FIG. 1A. A cell suspension, an orifice plate, a holder, a syringe, and a syringe pump used in each of Examples 1 to 6 are described below.

### (Preparation of Cell Suspension)

Cells used were Chinese hamster ovary cells (CHO-K1). Cells that were cultured on a plastic dish and became about 80% confluent were detached from a glass bottom dish with trypsin. A culture solution containing the cells collected from the glass bottom dish was centrifuged to remove a supernatant. After that, the cells were redispersed with Ham's F-12 Nutrient Mix (hereinafter referred to as “F-12 medium”) to provide a cell suspension.

A PBS solution containing fluorescein-labeled dextran (manufactured by Sigma-Aldrich, molecular weight: 70,000, hereinafter referred to as “FITC-Dex”) as a target substance at a concentration of 10 mg/ml was prepared. The PBS solution was added to the cell suspension prepared above to adjust the final concentration of the cells in the cell suspension to 0.5×10⁶ cells/mL and the final concentration of the FITC-Dex to 0.5 mg/mL.

The resultant cell suspension was filled into a countess cell counting chamber slide (manufactured by Thermo Fisher Scientific Inc.), and an image was recorded through use of a phase-contrast microscope (manufactured by Olympus Corporation, model number: CKX41). After that, 100 cells were measured for the diameter of a longest portion. The number-averaged cell diameter was 12.9 μm.

### (Orifice Plate)

A thin SUS plate with a diameter of 13 mm was used as the orifice plate, and a through hole serving as an orifice was formed at the center of the orifice plate by a laser. Observation of openings on both surfaces of the through hole by a digital microscope VHX-6000 manufactured by Keyence Corporation revealed that both the openings were perfectly circular. The diameters were also measured by the same device. The openings each had a perfect circular shape, and hence the size relationship of the areas of the inlet and the outlet of the orifice is the same size relationship of the diameters. In addition, the width of the narrowest portion of the orifice is equal to the diameter of the smaller opening, and the circle-equivalent diameter is equal to the diameter.

### (Holder)

A Swinnex filter holder, 13 mm manufactured by Millipore Corporation was used as the holder. The orifice plate was mounted on a filter mounting portion of the holder so that the opening with a smaller diameter of the orifice was on the inlet side. A through hole was formed in a central portion of a seating surface that supports the orifice plate so that the flow of the cell suspension from the orifice is not blocked.

### (Syringe)

A 2.5 mL gas-tight syringe 1002TLL manufactured by Hamilton Company was used as the syringe. 1.5 mL of the cell suspension was sucked into the syringe, and the syringe was coupled to the holder. The resultant was arranged so that the holder was placed in an upper portion, and the syringe was gently pushed out to discharge air from the orifice.

### (Syringe Pump)

PHD ULTRA manufactured by HARVARD was used as the syringe pump. The liquid feeding speed was set so as to achieve a predetermined flow velocity based on the smaller area of the orifice. The cell suspension that flowed out was received by a glass bottom dish with a diameter of 35 mm. The liquid feeding amount was set to 800 μL.

The diameter of the orifice, the distance from the inlet to the outlet of the orifice, and the flow velocity of the cell suspension at the inlet of the orifice in each of Examples are as shown in Table 1.

### (Evaluation)

An F-12 medium containing 10% serum was added to the glass bottom dish that has received the cell suspension, and the cells were incubated at 37° C. under an environment of 5% CO₂ for 2 hours. After that, the cells were detached from the glass bottom dish with trypsin and washed by centrifugation. Then, the cells were resuspended in PBS containing 2% serum. Subsequently, the fluorescence distribution of FITC was analyzed through use of flow cytometry (BD FACSMelody, BD Life Sciences-Biosciences). In addition, as a control, cells that were not passed through the orifice were also analyzed in the same manner, and the ratio of the cells exhibiting fluorescence intensity higher than that of the control was defined as the introduction rate of the target substance.

The evaluation results are shown in Table 1.

### Comparative Examples 1 to 6

In Comparative Examples 1 to 6, the cell processing and evaluation were performed in the same manner as in Examples 1 to 6 except that the orifice plates used in Examples 1 to 6 were each reversed and mounted on the holder so that the opening with a larger diameter of the orifice was placed at the inlet.

The evaluation results are shown in Table 1.

TABLE 1

| No. | | | | | | | | |
|---|---|---|---|---|---|---|---|---|
| of | | | | | | | | |
| Ex- | | | | | Distance | | Introduction | |
| ample/ | | | | | from | | rate (%) | |
| Com- | | | | | inlet to | | | Com- |
| para- | Diameter of | | Area of | | outlet | Flow | | para- |
| tive | opening of | | opening of | | of | velo- | | tive |
| Ex- | orifice (μm) | | orifice (μm²) | | orifice | city | Ex- | Ex- |
| ample | Small | Large | Small | Large | (μm) | (m/s) | ample | ample |
| 1 | 20 | 34 | 314 | 908 | 21 | 18 | 75 | 38 |
| 2 | 27 | 32 | 573 | 804 | 50 | 12 | 81 | 25 |
| 3 | 18 | 40 | 254 | 1,257 | 53 | 5 | 55 | 11 |
| 4 | 20 | 32 | 314 | 804 | 102 | 8 | 57 | 20 |
| 5 | 28 | 41 | 616 | 1,320 | 20 | 3 | 50 | 1 |
| 6 | 28 | 41 | 616 | 1,320 | 20 | 30 | 82 | 65 |

As is understood from the results of Examples and Comparative Examples, the introduction rate was able to be increased by causing the opening with a smaller diameter of the orifice to serve as the inlet. The high introduction rate of the FITC-Dex indicated that the cell processing, including the perforation of a cell membrane, is performed with high efficiency.

### Example 7

The cell processing was performed in the same manner as in Example 1 except that the target substance was changed to pDNA, pmaxGFP manufactured by Lonza K.K. The final concentration of the pmaxGFP was set to 0.25 μg/μL. Evaluation was performed in the same manner as in Example 1 except that the incubation time was changed to 1 day.

### Comparative Example 7

In Comparative Example 7, the cell processing and evaluation were performed in the same manner as in Example 7 except that the orifice plate used in Example 7 was reversed and mounted on the holder so that the opening with a larger diameter of the orifice was placed at the inlet.

The evaluation results of Example 7 and Comparative Example 7 are shown in Table 2.

TABLE 2

| No. | | | | | | | | |
|---|---|---|---|---|---|---|---|---|
| of | | | | | | | | |
| Ex- | | | | | | | Introduction | |
| ample/ | | | | | Distance | | rate (%) | |
| Com- | | | | | from | | | Com- |
| para- | Diameter of | | Area of | | inlet to | Flow | | para- |
| tive | opening of | | opening of | | outlet of | velo- | | tive |
| Ex- | orifice (μm) | | orifice (μm²) | | orifice | city | Ex- | Ex- |
| ample | Small | Large | Small | Large | (μm) | (m/s) | ample | ample |
| 7 | 20 | 34 | 314 | 908 | 21 | 18 | 32 | 7 |

Regarding the direction in which the orifice was mounted on the holder, in Example 7 in which the opening with a smaller diameter was caused to serve as the inlet, a higher introduction rate was obtained as compared to Comparative Example 7 in which the direction of the orifice was opposite to that in Example 7. From the foregoing, it was shown that high efficiency is obtained by the present invention also in the cell processing for introducing a gene.

As described above, in a cell processing method including a step of passing a liquid containing a cell through an orifice, the present invention was able to provide a cell processing method and a cell processing apparatus having high efficiency.

According to the present invention, a cell processing method and a cell processing apparatus having high efficiency of cell processing can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A cell processing method comprising a step of passing a liquid containing a cell through an orifice from a flow path by a pressure applying unit,
wherein the orifice is connected to the flow path so as to narrow a flow from the flow path, and
wherein the orifice includes at least any one of a portion in which an area of a cross-section perpendicular to a flow direction of the liquid passing through a center of the orifice is prevented from being changed from an inlet toward an outlet of the orifice or a portion in which the area is increased from the inlet toward the outlet of the orifice.

2. The cell processing method according to claim 1, which is to be used for perforating a cell membrane of the cell.

3. The cell processing method according to claim 1, which is to be used for introducing a target substance into the cell.

4. The cell processing method according to claim 3, wherein the target substance is a nucleic acid.

5. The cell processing method according to claim 3, wherein the target substance is a protein.

6. The cell processing method according to claim 3, wherein the target substance is a nonionic substance.

7. The cell processing method according to claim 3, wherein the liquid contains the target substance.

8. The cell processing method according to claim 1, which is to be used for lysing the cell.

9. The cell processing method according to claim 1, wherein a width of a narrowest portion in the cross-section is larger than a diameter of the cell.

10. The cell processing method according to claim 1, wherein an inlet circle-equivalent diameter of each of the portion in which the area is prevented from being changed from the inlet toward the outlet of the orifice and the portion in which the area is increased from the inlet toward the outlet of the orifice is 100 μm or less.

11. The cell processing method according to claim 1, wherein a distance from the inlet to the outlet of each of the portion in which the area is prevented from being changed from the inlet toward the outlet of the orifice and the portion in which the area is increased from the inlet toward the outlet of the orifice is 200 μm or less.

12. The cell processing method according to claim 1,
wherein the pressure applying unit is configured to enable a flow of the liquid directed from the flow path toward the orifice to be generated by operating a pressure generating unit, and
wherein the pressure generating unit is a pump.

13. The cell processing method according to claim 1, wherein the step of passing a liquid containing a cell through an orifice from a flow path by the pressure applying unit is performed through use of an inkjet type liquid ejection head.

14. The cell processing method according to claim 13, wherein the inkjet type liquid ejection head is a thermal inkjet type liquid ejection head.

15. A cell processing apparatus comprising:
an orifice forming member having an orifice;
a flow path connected to the orifice; and
a pressure applying unit,
wherein the pressure applying unit is configured to enable a flow of a liquid containing a cell directed from the flow path toward the orifice to be generated,
wherein the orifice is configured to narrow the flow of the liquid from the flow path, and
wherein the orifice includes at least any one of a portion in which an area of a cross-section perpendicular to a flow direction of the liquid passing through a center of the orifice is prevented from being changed from an inlet toward an outlet of the orifice or a portion in which the area is increased from the inlet toward the outlet of the orifice.

16. The cell processing apparatus according to claim 15, wherein a width of a narrowest portion of the cross-section is larger than a diameter of the cell.

17. The cell processing apparatus according to claim 15, wherein an inlet circle-equivalent diameter of each of the portion in which the area is prevented from being changed from the inlet toward the outlet of the orifice and the portion in which the area is increased from the inlet toward the outlet of the orifice is 100 μm or less.

18. The cell processing apparatus according to claim 15, wherein a distance from the inlet to the outlet of each of the portion in which the area is prevented from being changed from the inlet toward the outlet of the orifice and the portion in which the area is increased from the inlet toward the outlet of the orifice is 200 μm or less.

19. The cell processing apparatus according to claim 15, wherein the pressure generating unit is a pump.

20. The cell processing apparatus according to claim 15, which is an inkjet type liquid ejection head.

21. The cell processing apparatus according to claim 20, wherein the inkjet type liquid ejection head is a thermal inkjet type liquid ejection head.
